(19) United States
(12) Patent Application Publication
(10) Pub. No.: US 2004/0132803 A1
(43) Pub. Date: Jul. 8, 2004

(54) Agent for the treatment of overactive bladder

(76) Inventors: Tsuyoshi Yamagata, Shizuoka (JP); Kaoru Atsuki, Tokyo (JP); Tetsuji Ohno, Shizuoka (JP); Shiro Shirakura, Shizuoka (JP); Akira Karasawa, Shizuoka (JP); William C de Groat, Pittsburgh, PA (US); Naoki Yoshimura, Pittsburgh, PA (US); Adrian Sculptoreanu, Pittsburgh, PA (US)

Correspondence Address: FITZPATRICK CELLA HARPER & SCINTO, 30 ROCKEFELLER PLAZA, NEW YORK, NY, 10112, US

(21) Appl. No.: 10/473,418

(22) PCT Filed: Mar. 29, 2002
(86) PCT No.: PCT/US02/09577

(51) Int. Cl. A61K 31/38

(52) U.S. Cl. 514/431

(57) ABSTRACT

The present invention provides an agent for the treatment of overactive bladder, comprising, as an active ingredient, a compound having a slowly-inactivating A-type K⁺ channel opening activity or a pharmaceutically acceptable salt thereof, and a method for screening agents for the treatment of overactive bladder, comprising measuring a slowly-inactivating A-type K⁺ channel opening activity as an index.

### FIELD OF THE INVENTION

The present invention relates to agents for the treatment of overactive bladder.

### BACKGROUND OF THE INVENTION

Overactive bladder is a medical condition referring to the symptoms of urinary urgency and frequency, with or without urge urinary incontinence, when appearing in the absence of local pathologic or metabolic factors that would account for these symptoms. Storage and voiding of urine are physiologically controlled by complex reflex pathways including peripheral and central nervous systems (Urology, 50 Suppl. 6A: 36-52 (1997)). The urinary urgency refers to urgent and strong desire to void, and the urge urinary incontinence refers to involuntary urine leakage due to the urinary urgency.

In patients suffering from the symptoms such as urinary urgency and urge urinary incontinence due to overactive bladder, involuntary (uninhibited) contraction of the detrusor muscle is frequently observed in a cystometric measurement, and is called detrusor overactivity. This detrusor overactivity is considered to be a cause of urinary urgency and urge urinary incontinence. Moreover, urinary urgency can lead to urinary frequency. The detrusor overactivity is divided into 2 categories: neurogenic bladder (detrusor hyperreflex) when a neurological problem is found in a patient, and unstable bladder (detrusor instability) when a neurological problem is not found. It is considered that the cause of unstable bladder is potentially neurogenic bladder or disorder of detrusor smooth muscle per se (or both of them). Examples of the neurological problem relating to neurogenic bladder include Parkinson's disease, stroke, diabetes, multiple sclerosis, neuropathy and spinal cord injury.

Feeling of the filled bladder is transferred to the central nervous system via two bladder afferent neurons, the Aδ-fiber and the C-fiber; however, under the normal condition, the. C-fiber is not involved (silent). On the other hand, sensitivity of the C-fiber is known to be increased under the condition of bladder hypersensitivity and the like (Clinical J. Pain, 16: S86-89 (2000)). Furthermore, it is known that a spinal cord reflex mechanism via the C-fiber bladder afferents is involved in the overactive bladder in patients with supranuclear spinal cord injury (J. Urol., 157: 585-589 (1997)).

The potassium (K⁺) channel is present on cell membranes of various tissues and is involved in various physiological activities via the control of membrane potential. The K⁺ channel is classified into various types depending on the voltage-dependency, Ca⁺⁺-sensitivity, and other properties of the channel. The slowly-inactivating A-type K⁺ channel is expressed in capsaicin-sensitive dorsal root ganglion (DRG) neuronal cells (J. Neurophysiol., 75: 2629-2646 (1996)) and controls excitability of the C-fiber (J. Physiol., 494: 1-16 (1996)). The action potential of bladder afferent C-fiber of normal rats keeps a high threshold value by the effect of the slowly-inactivating A-type K⁺ channel. In contrast, in the rats with chronic cystitis, the K⁺ current is attenuated due to the changes in this ion channel characteristic. Thus, it has been supposed that at the time of cystitis excitability of the C-fiber increases, resulting in the overactive bladder (J. Neurosci., 19: 4644-4653 (1999)). In addition, in the rats with overactive bladder following spinal cord injury, density of the slowly-inactivating A-type K⁺ channel is reduced and excitability of the C-fiber is increased.

### DISCLOSURE OF THE INVENTION

According to the above observations, we have made the hypothesis that the overactive bladder, resulting from various diseases such as neurogenic bladder like spinal cord injury or bladder cystitis, can be treated by reducing excitability of the C-fiber through opening the slowly-inactivating A-type K⁺ channel. We have found a compound having a slowly-inactivating A-type K⁺ channel opening activity or a pharmaceutically acceptable salt thereof is useful for the treatment of overactive bladder, and we have achieved the present invention.

An object of the present invention is to provide an excellent agent for the treatment of overactive bladder.

The present invention relates to

(1) an agent for the treatment of overactive bladder, comprising, as an active ingredient, a compound having a slowly-inactivating A-type K⁺ channel opening activity or a pharmaceutically acceptable salt thereof, and

(2) the agent for the treatment of overactive bladder according to (1), wherein the compound having a slowly-inactivating A-type K⁺ channel opening activity is N-(5,5-dioxido-10-oxo-4,10-dihydrothieno[3,2-c][1]benzothiepin-9-yl)-3,3,3-trifluoro-2-hydroxy-2-methylpropanamide.

The present invention relates to

(3) a method for the treatment of overactive bladder, which comprises administering a therapeutically effective amount of a compound having a slowly-inactivating A-type K⁺ channel opening activity, or a pharmaceutically acceptable salt thereof, and

(4) use of a compound having a slowly-inactivating A-type K⁺ channel opening activity, or a pharmaceutically acceptable salt thereof for the manufacture of the agent for the treatment of overactive bladder.

Furthermore, the present invention relates to

(5) a method for screening agents for the treatment of overactive bladder, comprising measuring a slowly-inactivating A-type K⁺ channel opening activity as an index.

The term “compound having a slowly-inactivating A-type K⁺ channel opening activity” as used herein means all compounds having a slowly-inactivating A-type K⁺ channel opening activity regardless of a novel compound or a known compound and without limitation to the structure of compounds, so long as they have the slowly-inactivating A-type K⁺ channel opening activity as one of their properties.

The compounds having a slowly-inactivating A-type K⁺ channel opening activity used in the present invention include (S)-(+)-N-(5,5-dioxido-10-oxo-4,10-dihydrothieno[3,2-c][l]benzothiepin-9-yl)-3,3,3-trifluoro-2-hydroxy-2-methylpropanamide (Compound 1). Compound 1 is the same as Compound 1-25 described in WO 98/46587.

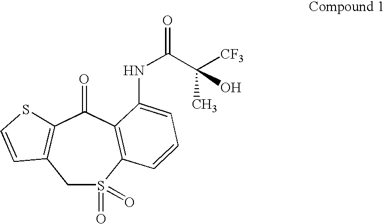

In the screening method of the present invention, the method for measuring a slowly-inactivating A-type K⁺ channel opening activity is not particularly limited, but examples thereof include methods descried in Test Examples 1 and 2 described below.

The pharmacological activities of the compound used in the present invention are described below based on Test Examples.

### TEST EXAMPLE 1

### Facilitatory effects on slowly-inactivating K⁺ Currents in DRG Cells

Materials and Methods

Animal Preparation:

Experiments were performed on adult female Sprague Dawley rats. First and second series of the experiments were performed, respectively, in unidentified DRG neurons and a specific population of DRG neurons innervating the urinary bladder. The population of DRG neurons that innervate the urinary bladder were labeled by retrograde axonal transport of the fluorescent dye, Fast Blue (4% w/v) (Polyloy, Gross Umstadt, Germany), injected into the wall of the bladder in halothane-anesthetized animals 7 days before the dissociation. The dye was injected with a 28 gauge needle at three to six sites on the dorsal surface of the organ (5-6 μL per site, total volume of 20-30 μL). Each injection site was washed with saline to minimize contamination of adjacent organs with the dye.

Cell Dissociation:

Freshly dissociated neurons from DRG were prepared from halothane-anesthetized animals. L6 and S1 DRG were dissected from animals and then dissociated in a shaking bath for 25 minutes at 35° C. with 5 mL DMEM (Sigma) containing 0.3 mg/mL trypsin (Type 3, Sigma), 1 mg/mL collagenase (Type 1, Sigma), and 0.1 mg/mL deoxyribonuclease (Type 4, Sigma). Trypsin inhibitor (Type 2a, Sigma) was then added thereto to neutralize the activity of trypsin. Individual DRG cell bodies were isolated by trituration and then plated on a poly-L-lysine-coated 35 mm Petri dishes.

Electrical Recordings:

Dye-labeled primary afferent neurons that innervate the urinary bladder were identified using an inverted phase-contrast microscope (Nikon, Tokyo, Japan) with fluorescent attachments (UV-1A filter; excitation wavelength, 365 nm). Gigaohm-seal whole-cell recordings were performed at room temperature (20-22° C.) on each labeled neuron in a culture dish that usually contained three to seven labeled cells among a few hundred unlabeled neurons. The internal solution contained (in mmol/L): KCl 140, CaCl₂ 1, MgCl₂ 2, EGTA 11, HEPES 10, Mg-ATP 2, and Tris-GTP 0.4 adjusted to pH 7.4 with KOH. Patch electrodes had resistances of 1-4 MΩ when filled with the internal solution. Neurons were superfused at a flow rate of 1.5 mL/minutes with an external solution containing (in mmol/L): NaCl 150, KCl 5, CaCl₂ 2.5, MgCl₂ 1, HEPES 10, and D-glucose 10, adjusted to pH 7.4 with NaOH. All recordings were made with an Axopatch-1D patch-clamp amplifier (Axon Instruments, Foster City, Calif.), and data were acquired and analyzed by PCLAMP software (Axon Instruments).

In voltage-clamp recordings, outward K⁺ currents and inward Na⁺ currents were measured. For the isolation of K⁺ currents, the external solution was changed to one containing (in mmol/L): choline-Cl 150, KOH 5, CaCl₂ 0.03, HEPES 10, Mg(OH)₂ 3, and D-glucose 10, adjusted to pH 7.4 with HCl.

In first series of experiments using unidentified DRG neurons, outward K⁺ currents were evoked by voltage steps to +60 mV, 800 ms long from a holding potentials of −90 mV, this was followed by a 1-second conditioning prepulse to −20 mV followed by a second pulse to +60 mV, identical to the first in the sequence. In the second series of experiments using Fast Blue-labeled bladder afferent neurons, slowly-inactivating A-type K⁺ currents were isolated by. subtraction of outward K⁺ currents activated from a holding potential of −40 mV (on the condition of inactivation of the majority of slowly-inactivating A-type K⁺ currents) from those activated from a holding potential of −120 mV (on the condition of full activation of slowly-inactivating A-type K⁺ currents). A compound was added cumulatively, starting with a lower concentration. Currents were measured at the maximum (peak) and normalized to control (before addition of the compound).

Inward Na⁺ currents were evoked by voltage steps to +60 mV, 800 ms long from a holding potential of −90 mV. Currents were measured at the maximum (peak) and normalized to control (before addition of the compound).

The results obtained in unidentified DRG neurons are shown in Tables 1 to 5, and the results obtained in capsaicin-sensitive bladder afferent neurons are shown in Table 6. Table 1 shows the activity of Compound 1 upon changes in currents when the holding potential is −90 mV (on the condition of activation of slowly-inactivating A-type K⁺ currents) or −20 mV (on the condition of inactivation of slowly-inactivating A-type K⁺ currents). Table 2 shows the activity of Compound 1 in the presence of 20 mmol/L tetraethylammonium, Table 3 shows the activity of Compound 1 in the presence of 5 μmol/L verapamil, and Table 4 shows the activity of Compound 1 in the presence of 60 mmol/L tetraethylammonium. Table 5 shows the activity of Compound 1 upon Na⁺ currents. In Tables 1 to 5, n means the number of cases.

TABLE 1

| Compound 1 | | | | |
|---|---|---|---|---|
| (mol/L) | HP: −90 mV | n | HP: −20 mV | n |
| 5.0 × 10⁻⁹ | 1.01 ± 0.03 | 7 | 0.98 ± 0.02 | 7 |
| 1.0 × 10⁻⁸ | 0.99 ± 0.08 | 3 | 0.98 ± 0.04 | 3 |
| 2.5 × 10⁻⁸ | 1.07 ± 0.03 | 5 | 1.01 ± 0.03 | 5 |
| 5.0 × 10⁻⁸ | 1.07 ± 0.02 | 16 | 1.00 ± 0.01 | 16 |
| 1.0 × 10⁻⁷ | 1.12 ± 0.03 | 10 | 1.02 ± 0.01 | 10 |
| 5.0 × 10⁻⁷ | 1.15 ± 0.03 | 7 | 1.01 ± 0.02 | 7 |
| 1.0 × 10⁻⁶ | 1.06 ± 0.02 | 13 | 1.00 ± 0.02 | 13 |
| 1.0 × 10⁻⁵ | 0.96 ± 0.05 | 8 | 0.88 ± 0.07 | 8 |
| 5.0 × 10⁻⁵ | 0.83 ± 0.09 | 3 | 0.81 ± 0.08 | 3 |

Table 1 shows the activity of Compound 1 on currents measured by a voltage clamp using unidentified DRG cells. The currents at a holding potential (HP) of −90 mV are results of the measurement of slowly-inactivating A-type K⁺ currents, and the currents at an HP value of −20 mV are results of the measurement of delayed rectifier K⁺ currents. Table 1 shows that Compound 1 increases slowly-inactivating A-type K⁺ currents with a peak compound concentration of from 1×10⁻⁷ to 5×10⁻⁷ mol/L, but does not have no influences (little influences) upon delayed rectifier K⁺ currents. Also, effects (values) of Compound 1 are shown by relative values when the values before the compound application are defined as 1.

TABLE 2

| Compound 1 | | | | |
|---|---|---|---|---|
| (mol/L) | HP: −90 mV | N | HP: −20 mV | n |
| 5.0 × 10⁻⁹ | 1.02 ± 0.02 | 8 | 0.99 ± 0.00 | 8 |
| 5.0 × 10⁻⁸ | 1.08 ± 0.02 | 10 | 0.99 ± 0.01 | 10 |
| 1.0 × 10⁻⁷ | 1.10 ± 0.01 | 3 | 0.99 ± 0.02 | 3 |
| 5.0 × 10⁻⁷ | 1.15 ± 0.04 | 14 | 1.01 ± 0.01 | 14 |
| 1.0 × 10⁻⁶ | 1.19 ± 0.05 | 4 | 1.00 ± 0.00 | 4 |
| 5.0 × 10⁻⁶ | 1.16 ± 0.03 | 10 | 0.98 ± 0.01 | 10 |
| 1.0 × 10⁻⁵ | 1.19 ± 0.01 | 3 | 1.01 ± 0.02 | 3 |
| 5.0 × 10⁻⁵ | 0.91 ± 0.04 | 8 | 0.84 ± 0.06 | 8 |
| 5.0 × 10⁻⁴ | 0.29 ± 0.02 | 4 | 0.25 ± 0.04 | 4 |

Table 2 shows the activity of Compound 1 in the presence of tetraethylammonium as a blocker of delayed rectifier K⁺ currents. Since Compound 1 shows the same results in the presence of the blocker of delayed rectifier K⁺ currents, it is suggested that the K⁺ currents increasing effect of Compound 1 is not mediated by the delayed rectifier K⁺ channel.

TABLE 3

| Compound 1 | | | | |
|---|---|---|---|---|
| (mol/L) | HP: −90 mV | N | HP: −20 mV | n |
| 5.0 × 10⁻⁸ | 1.03 ± 0.02 | 3 | 1.00 ± 0.00 | 3 |
| 1.0 × 10⁻⁷ | 1.13 ± 0.02 | 4 | 1.00 ± 0.01 | 4 |
| 5.0 × 10⁻⁷ | 1.17 ± 0.03 | 8 | 1.00 ± 0.01 | 8 |
| 1.0 × 10⁻⁶ | 1.11 ± 0.03 | 5 | 1.00 ± 0.01 | 5 |
| 5.0 × 10⁻⁶ | 1.02 ± 0.03 | 6 | 1.01 ± 0.01 | 6 |
| 1.0 × 10⁻⁵ | 0.99 ± 0.02 | 6 | 1.00 ± 0.02 | 6 |
| 5.0 × 10⁻⁵ | 0.94 ± 0.02 | 9 | 0.98 ± 0.01 | 9 |
| 5.0 × 10⁻⁴ | 0.61 ± 0.05 | 6 | 0.83 ± 0.06 | 6 |

Table 3 shows the activity of Compound 1 in the presence of verapamil as a blocker of delayed rectifier K⁺ currents. Since the results obtained in Table 3 are similar to those in Table 2, it is suggested that the K⁺ currents increasing effect of Compound 1 is not mediated by the delayed rectifier K⁺ channel.

TABLE 4

| Compound 1 | | | | |
|---|---|---|---|---|
| (mol/L) | HP: −90 mV | N | HP: −20 mV | n |
| 5.0 × 10⁻⁸ | 1.08 ± 0.02 | 12 | 1.00 ± 0.01 | 12 |
| 5.0 × 10⁻⁷ | 1.11 ± 0.03 | 12 | 0.97 ± 0.03 | 12 |
| 5.0 × 10⁻⁶ | 1.14 ± 0.05 | 10 | 0.97 ± 0.02 | 10 |
| 5.0 × 10⁻⁵ | 1.15 ± 0.08 | 7 | 0.95 ± 0.03 | 7 |
| 5.0 × 10⁻⁴ | 0.56 ± 0.05 | 5 | 0.54 ± 0.02 | 5 |

Table 4 shows the activity of Compound 1 in the presence of high concentration tetraethylammonium (TEA). The high concentration tetraethylammonium (TEA) acts as a blocker of delayed rectifier K⁺ currents. Since the results obtained in Table 4 are also similar to those in Table 2, it is suggested that the K⁺ currents increasing effect of Compound 1 is not mediated by the delayed rectifier K⁺ channel.

TABLE 5

| Compound 1 | | |
|---|---|---|
| (mol/L) | Na⁺ ion currents | n |
| 5.0 × 10⁻⁸ | 0.99 ± 0.01 | 9 |
| 5.0 × 10⁻⁷ | 0.99 ± 0.01 | 9 |
| 1.0 × 10⁻⁶ | 0.99 ± 0.02 | 4 |
| 5.0 × 10⁻⁶ | 1.00 ± 0.00 | 6 |
| 5.0 × 10⁻⁵ | 1.00 ± 0.01 | 6 |
| 5.0 × 10⁻⁴ | 1.00 ± 0.01 | 5 |

Table 5 shows the activity of Compound 1 upon Na⁺ currents in DRG cells. It is evident from Table 5 that Compound 1 does not exert influence upon Na⁺ currents.

TABLE 6

| | | | Delayed | |
|---|---|---|---|---|
| Compound 1 | A-type I_{K} | | rectifier | |
| (mol/L) | (HP: −120 mV)-(HP: −40 mV) | n | HP: −40 mV | n |
| 1.0 × 10⁻⁶ | 1.26 ± 0.03 | 6 | 1.10 ± 0.01 | 6 |

Table 6 shows the activity of Compound 1 upon slowly-inactivating A-type K⁺ currents (I_{K}) and delayed rectifier K⁺ currents in Fast Blue-labeled bladder afferent neurons that were sensitive to capsaicin (presumed C-fiber neurons). Slowly-inactivating A-type K⁺ currents were isolated by subtraction of outward K⁺ currents activated from a holding potential of −40 mV (on the condition of inactivation of the majority of slowly-inactivating A-type K⁺ currents) from those activated from a holding potential of −120 mV (on the condition of full activation of slowly-inactivating A-type K⁺ currents). As demonstrated in unidentified DRG neurons, Table 6 shows that Compound 1 increases slowly-inactivating A-type K⁺ currents, but have smaller influences upon delayed rectifier K⁺ currents.

### TEST EXAMPLE 2

### Changes in Membrane Potential in DRG Cells

Material and Methods

Animal Preparation:

Experiments were performed on adult female Sprague Dawley rats. A population of unidentified DRG cells and a population of DRG neurons that innervate the urinary bladder were labeled by retrograde axonal transport of the fluorescent dye, Fast Blue (4% w/v) (Polyloy, Gross Umstadt, Germany), injected into the wall of the bladder in halothane-anesthetized animals 7 days before the dissociation. The dye was injected with a 28 gauge needle at three to six sites on the dorsal surface of the organ (5-6 μL per site, total volume of 20-30 μL). Each injection site was washed with saline to minimize contamination of adjacent organs with the dye.

Cell Dissociation:

Freshly dissociated neurons from DRG were prepared from halothane-anesthetized animals. L6 and S1 DRG were dissected from animals and then dissociated in a shaking bath for 25 minutes at 35° C. with 5 mL DMEM (Sigma) containing 0.3 mg/mL trypsin (Type 3, Sigma), 1 mg/mL collagenase (Type 1, Sigma), and 0.1 mg/mL deoxyribonuclease (Type 4, Sigma). Trypsin inhibitor (Type 2a, Sigma) was then added thereto to neutralize the activity of trypsin. Individual DRG cell bodies were isolated by trituration and then plated on a poly-L-lysine-coated 35 mm Petri dishes.

Electrical Recordings:

Dye-labeled primary afferent neurons that innervate the urinary bladder were identified using an inverted phase-contrast microscope (Nikon, Tokyo, Japan) with fluorescent attachments (UV-1A filter; excitation wavelength, 365 nm). Gigaohm-seal whole-cell recordings were performed within 6-8 hours after cell dissociation at room temperature (20-22° C.) on each labeled neuron in a culture dish that usually contained three to seven labeled cells among a few hundred unlabeled neurons. The internal solution contained (in mmol/L): KCl 140, CaCl₂ 1, MgCl₂ 2, EGTA 11, HEPES 10, Mg-ATP 2, and Tris-GTP 0.4 adjusted to pH 7.4 with KOH. Patch electrodes had resistances of 1-4 M Ω when filled with the internal solution. Neurons were superfused at a flow rate of 1.5 mL/minutes with an external solution containing (in mmol/L): NaCl 150, KCl 5, CaCl₂ 2.5, MgCl₂ 1, HEPES 10, and D-glucose 10, adjusted to pH 7.4 with NaOH. All recordings were made with an Axopatch-1D patch-clamp amplifier (Axon Instruments, Foster City, Calif.), and data were acquired and analyzed by PCLAMP software (Axon Instruments).

In current-clamp recordings, membrane potential of DRG cells were measured before and after compound applications. The membrane potentials were normalized to control (before addition of the compound).

Effect of Compound 1 on membrane potential is shown in Table 7.

TABLE 7

| Compound 1 | | |
|---|---|---|
| (mol/L) | Membrane potential (mV) | n |
| 0 | −47.95 ± 0.22 | 20 |
| 1.0 × 10⁻⁹ | −48.10 ± 0.48 | 10 |
| 1.0 × 10⁻⁸ | −49.80 ± 0.65 | 5 |
| 1.0 × 10⁻⁷ | −57.17 ± 0.53 | 6 |
| 1.0 × 10⁻⁶ | −53.67 ± 0.74 | 6 |
| 5.0 × 10⁻⁶ | −50.50 ± 0.88 | 4 |
| 5.0 × 10⁻⁵ | −48.50 ± 1.28 | 4 |
| 5.0 × 10⁻⁴ | −42.67 ± 1.46 | 3 |

Table 7 shows the activity of Compound 1 upon membrane potential in DRG cells. It showed that Compound 1 increases slowly-inactivating A-type K⁺ currents, namely increases outward currents to effect hyperpolarization (a negative change of membrane potential). This activity suggests reduction of excitability of DRG cells.

Based on the results of Test Examples 1 and 2, it was revealed that Compound 1 has an activity of increasing slowly-inactivating A-type K⁺ currents.

### TEST EXAMPLE 3

### Activity of Inhibiting Detrusor Hyperreflexia

The test was carried out in accordance with the method of Cheng et al. (Brain Res., 678: 40-48 (1995)).

Female SD rats of 8 to 10 weeks of age (supplied by Japan SLC) were used in the test. Five to seven animals of these rats were put in each metal cage and reared by allowing them to freely take commercially available chow and water, in a rearing room at a room temperature of from 19 to 25° C. and a humidity of from 30 to 70% under illumination for 12 hours (from 7 a.m. to 7 p.m.) per day.

Spinal cord injury was induced in rats. Each rat was anesthetized with diethyl ether and the skin of the backside thoracic cord part was incised. Vertebral arch around the 7th to 8th thoracic vertebrae was excised in a length of about 5 mm, and the wound cavity of the excised part was filled with cellulose oxide for blood stanching. The incised part was sutured with a surgical silk thread. After the spinal cord injury operation, forced-pressure urination was manually carried out for about 3 weeks twice a day (between 8 and 9 o'clock and 18 and 19 o'clock) until complete automatic micturition developed. Also, intramuscular injection of the antibiotic ampicillin (manufactured by Sigma, 150 mg/kg) was carried out once or twice a day for about 2 weeks.

Four to five weeks after the spinal cord injury, each rat was subjected to bladder catheter operation. The bladder was exposed by midline incision of the abdomen under diethyl ether anesthesia. A polyethylene tube (PE-50; Becton Dickinson) having a blunt end to protect tissues from injury was filled with physiological saline (Otsuka Pharmaceutical Industries, Tokushima, Japan) and inserted from the bladder top. This bladder catheter was fixed with a surgical silk thread and indwelled. Also, the other end was exposed subcutaneously from the back neck, plugged and then fixed to the skin with the surgical thread.

Four to six days after the bladder catheter operation, a cystometry test was carried out. The rat was put in a Ballman cage (Natsume Seisakusho), a three way cock was connected to the bladder catheter, one end of the cock was connected to a pressure transducer (Nihon Kohden) and the other end was connected to a 50 mL capacity syringe (Terumo) arranged to an infusion pump (Harvard Apparatus) for physiological saline injection. The intravesical pressure signal from the pressure transducer was amplified by a strain pressure amplifier (AP-601G; Nihon Kohden) connected thereto and recorded on a thermal array recorder (RTA-1200; Nihon Kohden) via a polygraph system (RPM-6008; Nihon Kohden) contained therein. Sixty to ninety minutes after the completion of the preparation, physiological saline kept at room temperature was continuously injected into the bladder at a flow rate of 10 mL/h for 30 minutes, and the occurrence of micturition contraction was confirmed. Thirty minutes after the treatment, physiological saline was injected again over 30 minutes, and the intravesical pressure was measured to be used as a pre-drug administration value. The test compound (Compound 1) was suspended in 0.5 w/v % aqueous methyl cellulose at a concentration of 1 mg/mL. This suspension was further diluted with 0.5 w/v % aqueous methyl cellulose to prepare a suspension or solution for the administration at the intended concentration, and orally administered at a volume of 1 mL/kg. The period of 1, 3 or 5 hours after the administration was used as the measuring time after the administration of the solvent or the drug tested, and the intravesical injection of physiological saline was carried out during a duration of 15 minutes around each measuring time (45 to 75 minutes, 165 to 195 minutes and 285to 315 minutes after the administration of the drug).

Micturition contraction was measured as the index of normal voiding function, and pre-micturition contraction as the index of detrusor hyperreflexia. The average of all micturition contraction values observed during each of the 30 minutes-measuring periods and the average of maximum pre-micturition contraction values observed during each micturition contraction period were respectively defined as the size of micturition contraction and pre-micturition contraction at each period. In this case, both of the contraction values were read out from the intravesical pressure wave form recorded on the chart paper, using a digitizer (KD3220; Graphtech) controlled by a computer (PC-9801NS/R; manufactured by NEC), and stored as a WJ2 type file on Lotus 1-2-3 R2.5J (manufactured by Lotus). The WJ2 file was put in Excel for Windows version 7.0 (manufactured by Microsoft). Sizes of pre-micturition contraction and micturition contraction were converted to relative values when the values before the drug administration was defined as 100, and average± standard error was calculated for each group.

The results for Compound 1 are shown in Table 8 on the value (%) of pre-micturition contraction after the administration of the solvent or agent, and in Table 9 on the value (%) of micturition contraction.

TABLE 8

| | | Compound 1 (mg/kg, p.o.) | | |
|---|---|---|---|---|
| | Control | 0.001 | 0.01 | 0.1 |
| Before | 100.0 ± 0.0 | 100.0 ± 0.0 | 100.0 ± 0.0 | 100.0 ± 0.0 |
| administration | | | | |
| After 1 hour | 115.0 ± 12.1 | 83.5 ± 6.8 | 59.6 ± 8.1* | 52.3 ± 9.1* |
| After 3 hours | 128.4 ± 21.5 | 95.4 ± 12.0 | 51.2 ± 6.7* | 33.3 ± 6.3* |
| After 5 hours | 120.2 ± 24.5 | 105.5 ± 20.8 | 42.2 ± 7.2* | 28.5 ± 6.2* |

*p < 0.05 (comparison with the control group)
(n = 5-6; Dunnett's test)

TABLE 9

| | | Compound 1 (mg/kg, p.o.) | | |
|---|---|---|---|---|
| | Control | 0.001 | 0.01 | 0.1 |
| Before | 100.0 ± 0.0 | 100.0 ± 0.0 | 100.0 ± 0.0 | 100.0 ± 0.0 |
| administration | | | | |
| After 1 hour | 97.5 ± 5.9 | 106.5 ± 11.3 | 114.4 ± 9.5 | 109.3 ± 6.3 |
| After 3 hours | 99.3 ± 4.8 | 101.7 ± 9.5 | 117.6 ± 13.9 | 110.2 ± 5.4 |
| After 5 hours | 94.2 ± 6.5 | 103.1 ± 6.5 | 117.6 ± 12.7 | 112.4 ± 7.0 |

According to the results shown in Test Example 3, Compound 1 inhibited pre-micturition contraction (detrusor overactivity) in spinal cord-injured rats, but had no influence on micturition (physiological) contraction.

### TEST EXAMPLE 4

### Activity of Inhibiting Detrusor Instability

The test was carried out in accordance with the method of Malmgren et al. (J. Urol., 142: 1134-1138 (1989)).

Female SD rats of 8 to 10 weeks of age (supplied by Japan SLC) were used in the test. Five to seven animals of these rats were put in each metal cage and reared by allowing them to freely take commercially available solid chow and water, in a rearing room at a room temperature of from 19 to 25° C. and a humidity of from 30 to 70% under illumination for 12 hours (from 7 a.m. to 7 p.m.) per day.

Partial urethra obstruction was induced in rats. Each rat was anesthetized by intraperitoneal administration of 50 mg/kg of pentobarbital sodium (Dainippon Pharmaceutical, Osaka, Japan) and the skin and muscle of the abdominal side were cut by midline incision. A polyethylene tube (PE-20; Becton Dickinson) was inserted into urethra. The urethra base was peeled and double-ligated, and then the polyethylene tube was pulled out to induce partial obstruction of the urethra. The incised part was sutured with a surgical silk thread. The antibiotic ampicillin (manufactured by Sigma, 150 mg/kg) was intramuscularly injected.

Six weeks after the urethra obstruction operation, each rat with hypertrophic bladder was subjected to bladder catheter operation. The bladder was exposed by midline incision of the abdomen under pentobarbital sodium anesthesia. A polyethylene tube (PE-50; Becton Dickinson) having a blunt end to protect tissues from injury was filled with physiological saline (Otsuka Pharmaceutical Industries, Tokushima, Japan) and inserted from the bladder top. This bladder catheter was fixed with a surgical silk thread and indwelled. The other end was exposed subcutaneously from the back neck, plugged and then fixed to the skin with the surgical silk thread.

Four to six days after the bladder catheter operation, a cystometry test was carried out. The rat was put in a Ballman cage (Natsume Seisakusho), a three way cock was connected to the bladder catheter, one end of the cock was connected to a pressure transducer (Nihon Kohden) and the other end was connected to a 50 mL capacity syringe (Terumo) arranged to an infusion pump (Harvard Apparatus) for physiological saline injection. The intravesical pressure signal from the pressure transducer was amplified by a strain pressure amplifier (AP-601G; Nihon Kohden) connected thereto and recorded on a thermal array recorder (RTA-1200; Nihon Kohden) via a polygraph system (RPM-6008; Nihon Kohden) contained therein. Sixty to ninety minutes after the completion of the preparation, physiological saline kept at room temperature was continuously injected into the bladder at a flow rate of 10 mL/h until the completion of the test, and the occurrence of micturition contraction and pre-urination contraction were confirmed. Charts for 30 minutes after 3 hours from -the commencement of the physiological saline injection were used as the values before the drug administration. The test compound was suspended in 0.5 w/v % aqueous methyl cellulose at a concentration of 1 mg/mL. This suspension was further diluted with 0.5 w/v % aqueous methyl cellulose to prepare a suspension or solution for the administration at the intended concentration. This was orally administered at a volume of 1 mL/kg. The period of 1, 3 or 5 hours after the administration was used as the measuring time after the administration of the drug tested, and a duration of 15 minutes around each measuring time (45 to 75 minutes, 165 to 195 minutes and 285 to 315 minutes after the administration of the drug) was used as the measuring period.

Micturition contraction was measured as the index of normal voiding function, and pre-micturition contraction as the index of detrusor instability. The average of all micturition contraction values observed during each of the 30 minutes-measuring periods and the average of maximum pre-micturition contraction values observed during each micturition contraction period were respectively defined as the size of micturition contraction and pre-micturition contraction at each period. In this case, both of the contraction values were read out from the intravesical pressure wave form recorded, on the chart paper, using a digitizer (KD3220; Graphtech) controlled by a computer (PC-9801NS/R; manufactured by NEC), and stored as a WJ2 type file on Lotus 1-2-3 R2.5J (manufactured by Lotus). The WJ2 file was put in Excel for Windows version 7.0 (manufactured by Microsoft). Sizes of pre-micturition contraction and micturition contraction were converted to relative values when the values before the drug administration was defined as 100, and average ± standard error was calculated for each group.

Table 10 shows the value (%) of pre-micturition contraction after the administration of the solvent or Compound 1, and Table 11 shows the value (%) of micturition contraction after the administration of the solvent or Compound 1.

TABLE 10

| | | Compound 1 (mg/kg, p.o.) | | |
|---|---|---|---|---|
| | Control | 0.001 | 0.01 | 0.1 |
| Before | 100.0 ± 0.0 | 100.0 ± 0.0 | 100.0 ± 0.0 | 100.0 ± 0.0 |
| administration | | | | |
| After 1 hour | 105.5 ± 6.5 | 105.9 ± 7.8 | 59.5 ± 4.1*** | 55.8 ± 8.2*** |
| After 3 hours | 109.4 ± 14.8 | 100.3 ± 10.0 | 69.5 ± 4.4* | 66.2 ± 4.7* |
| After 5 hours | 103.3 ± 3.6 | 104.2 ± 10.2 | 67.8 ± 5.5** | 69.2 ± 7.4** |

*p < 0.05,
**p < 0.01,
***p < 0.001 (comparison with the control group)
(n = 5-6, Dunnett's test)

TABLE 11

| | | Compound 1 (mg/kg, p.o.) | | |
|---|---|---|---|---|
| | Control | 0.001 | 0.01 | 0.1 |
| Before | 100.0 ± 0.0 | 100.0 ± 0.0 | 100.0 ± 0.0 | 100.0 ± 0.0 |
| administration | | | | |
| After 1 hour | 102.5 ± 3.5 | 99.3 ± 2.3 | 99.6 ± 1.2 | 106.2 ± 9.2 |
| After 3 hours | 104.4 ± 5.1 | 101.5 ± 3.3 | 92.6 ± 3.2 | 105.2 ± 9.6 |
| After 5 hours | 96.6 ± 3.2 | 98.7 ± 5.2 | 90.1 ± 4.3 | 100.1 ± 8.6 |

According to the results shown in Test Example 4, Compound 1 had no influence on micturition contraction which is the contraction at micturition in rats with hypertrophic bladder (no influence on normal voiding), but inhibited pre-micturition contraction which is irregular, detrusor instability at the time other than normal voiding.

Test Examples 3 and 4 show that the compound used in the present invention inhibits the pre-urination contraction (detrusor overactivity) and are useful as an agent for the treatment of overactive bladder. Thus, it is considered that the compound having a slowly-inactivating A-type K⁺ channel opening activity or a pharmaceutically acceptable salt thereof is useful as an agent for the treatment of overactive bladder.

### TEST EXAMPLE 5

### Acute Toxicity Test

The test compound was administered orally or intraperitoneally to 3 animals per group of dd male mice (body weight, 20±1 g). Minimum lethal dose (MLD) value was obtained by observing mortality on the 7th day after the administration.

As a result, MLD of Compound 1 was >1,000 mg/kg by oral administration.

Based on the results of Test Examples 1 to 5, the compound having a slowly-inactivating A-type K⁺ channel opening activity or a pharmaceutically acceptable salt thereof is useful as an agent for the treatment of overactive bladder.

The compound having a slowly-inactivating A-type K⁺ channel opening activity or a pharmaceutically acceptable salt thereof can be used as it is or in various dose forms. Pharmaceutical compositions of the present invention can be produced by uniformly mixing an effective amount of the compound having a slowly-inactivating A-type K⁺ channel opening activity or a pharmaceutically acceptable salt thereof as an active ingredient with a pharmacologically acceptable carrier. It is preferred that these pharmaceutical compositions are in a unit dose form suitable for oral or parenteral (including intravenous) administration or the like.

In preparing a composition in the oral dose form, certain useful pharmacologically acceptable carriers can be used. For example, oral liquid preparations such as suspensions or syrups can be produced using water; saccharides, such as sucrose, sorbitol, fructose, or the like; glycols, such as polyethylene glycol, propylene glycol, or the like; oils, such as sesame oil, olive oil, soybean oil, or the like; antiseptics, such as p-hydroxybenzoic acid esters or the like; flavors, such as strawberry flavor, peppermint, or the like; or the like. Capsules, tablets, powders and granules can be produced using fillers, such as lactose, glucose, sucrose, mannitol, or the like; disintegrators, such as starch, sodium alginate, or the like; lubricants, such as magnesium stearate, talc, or the like; binders, such as polyvinyl alcohol, hydroxypropylcellulose, gelatin, or the like; surfactants, such as fatty acid esters or the like; plasticizers, such as glycerine or the like; or the like. Tablets and capsules are the most useful unit oral administration preparations because of their easy administration. In producing tablets or capsules, solid pharmaceutical carriers are used.

In addition, injections can be prepared using a carrier comprising distilled water, a salt solution, a glucose solution or a mixture of salt water and a glucose solution. In this case, they are prepared as solutions, suspensions or dispersions using suitable auxiliaries in the conventional way.

The compound having a slowly-inactivating A-type K⁺ channel opening activity or a pharmaceutically acceptable salt thereof can be administered orally in the above dose forms or parenterally as injections, and, although its effective dose and administration frequency may vary depending, for example, on the dose form, the age and body weight of each patient, and symptoms of the disease, from 1 to 900 mg/60 kg/day, preferably from 1 to 200 mg/60 kg/day, is suitable.

The embodiments of the present invention are described below based on Examples.

### BEST MODE FOR CARRYING OUT THE INVENTION

### EXAMPLE 1

### Tablets

Tablets having the following composition were prepared in the conventional way.

Compound 1 (250 g) was mixed with 1598.5 g of mannitol, 100 g of sodium starch glycollate, 10 g of light anhydrous silicic acid, 40 g of magnesium stearate and 1.5 of yellow ferric oxide in the conventional way. The resulting mixture was applied to a tablet making machine having a punch and die of 8 mm in diameter (Purepress Correct-12, manufactured by Kikusui) to obtain tablets (containing 25 mg of the active component per one tablet).

The prescription is shown in Table 12.

TABLE 12

| | | | |
|---|---|---|---|
| Prescription | Compound 1 | 25 | mg |
| | Mannitol | 159.85 | mg |
| | Sodium starch glycollate | 10 | mg |
| | Light anhydrous silicic acid | 1 | mg |
| | Magnesium stearate | 4 | mg |
| | Yellow ferric oxide | 0.15 | mg |
| | | 200 | mg |

### EXAMPLE 2

### Capsules

Capsules having the following composition were prepared in the conventional way.

Compound 1 (500 g) was mixed with 300 g of lactose, 100 g of light anhydrous silicic acid and 100 g of sodium lauryl sulfate in the conventional way. The resulting mixture was packed in hard capsules No. 1 (100 mg per one capsule) using an encapsulation machine (LZ-64, manufactured by Zanasi) to obtain capsules (containing 50 mg of the active component per one capsule).

The prescription is shown in Table 13.

TABLE 13

| | | | |
|---|---|---|---|
| Prescription | Compound 1 | 50 | mg |
| | Lactose | 30 | mg |
| | Light anhydrous silicic acid | 10 | mg |
| | Sodium lauryl sulfate | 10 | mg |
| | | 100 | mg |

### EXAMPLE 3

### Injections

Injections having the following composition are prepared in the conventional way.

Compound 1 (1 g) is dissolved in 100 g of purified soybean oil, and 12 g of purified yolk lecithin and 25 g of glycerol for injection are added thereto. The resulting mixture is kneaded with distilled water for injection (total: 1,000 mL) and emulsified therein in the conventional way. The obtained dispersion is aseptically filtered using a 0.2 μm disposable membrane filter and then aseptically dispensed into glass vials in 2 ml portions to obtain injections (containing 2 mg of the active component per one vial).

The prescription is shown in Table 14.

TABLE 14

| | | | |
|---|---|---|---|
| Prescription | Compound 1 | 2 | mg |
| | Purified soybean oil | 200 | mg |
| | Purified yolk lecithin | 24 | mg |
| | Glycerol for injection | 50 | mg |
| | Distilled water for injection | 1.72 | ml |
| | | 2.00 | ml |

### Industrial Applicability

The present invention provides an agent for the treatment of overactive bladder, comprising, as an active ingredient, a compound having a slowly-inactivating A-type K⁺ channel opening activity or a pharmaceutically acceptable salt thereof, and a method for screening agents for the treatment of overactive bladder, comprising measuring a slowly-inactivating A-type K⁺ channel opening activity as an index.

1. An agent for the treatment of overactive bladder, comprising, as an active ingredient, a compound having a slowly-inactivating A-type K⁺ channel opening activity or a pharmaceutically acceptable salt thereof.

2. The agent for the treatment of overactive bladder according to claim 1, wherein the compound having a slowly-inactivating A-type K⁺ channel opening activity is N-(5,5-dioxido-10-oxo-4,10-dihydrothieno[3,2-c][1]benzothiepin-9-yl)-3,3,3-trifluoro-2-hydroxy-2-methylpropanamide.

3. A method for the treatment of overactive bladder, which comprises administering a therapeutically effective amount of a compound having a slowly-inactivating A-type K⁺ channel opening activity, or a pharmaceutically acceptable salt thereof.

4. Use of a compound having a slowly-inactivating A-type K⁺ channel opening activity, or a pharmaceutically acceptable salt thereof for the manufacture of the agent for the treatment of overactive bladder.

5. A method for screening agents for the treatment of overactive bladder, comprising measuring a slowly-inactivating A-type K⁺ channel opening activity as an index.
